(19) United States
(12) Patent Application Publication
(10) Pub. No.: US 2008/0171969 A1
(43) Pub. Date: Jul. 17, 2008

(54) Syringe

(75) Inventors: Phillip Owen Byrne, Newcastle upon Tyne (GB); Harry Raymond Ingham, Newcastle upon Tyne (GB); Penelope Rosemary Attridge, Middlesex (GB)

Correspondence Address: NIXON & VANDERHYE, PC, 901 NORTH GLEBE ROAD, 11TH FLOOR, ARLINGTON, VA, 22203, US

(73) Assignee: ID-Tech Limited, Newcastle Upon Tyne (GB)

(21) Appl. No.: 10/583,451

(22) PCT Filed: Dec. 20, 2004
(86) PCT No.: PCT/GB2004/005357

§ 371 Date: Aug. 3, 2006

(30) Foreign Application Priority Data
Dec. 18, 2003 (GB) 0329269.5

(51) Int. Cl. A61M 37/00; A61M 5/00; A61M 1/06

(52) U.S. Cl. 604/73; 604/141; 604/187

(57) ABSTRACT

A disposable syringe (2) is disclosed. Withdrawal of a plunger handle (18) in the direction of arrow B to fill the syringe causes a moveable safety bobbin (20) to lock, and shaft (16) slides in the direction of arrow B relative to safety bobbin (20) and washer (22). Subsequent depression of plunger handle (18) in the direction of arrow A causes safety bobbin (20) to move along barrel (4), from which position it can not be withdrawn. This therefore prevents subsequent withdrawal of piston (14) and therefore prevents re-use of the syringe.

### TECHNICAL FIELD

The present invention relates to single use syringes, that is to say syringes which are intended to be used once and which are adapted in some way to prevent or at least to hinder further use.

### BACKGROUND ART

Disposable syringes are known in which a cylindrical barrel formed of transparent plastics material receives a piston which is slidable within the barrel. A shaft, which may be of cruciform or other, e.g. circular, section extends from the piston to a plunger handle for enabling the piston to be displaced along the barrel in a first or proximal direction to cause injectable fluid or body fluid to be drawn into the barrel via an aperture at one end of the barrel, or in a second or distal direction to cause the fluid to be expelled out of the aperture or to be injected into a patient via a needle.

Syringes of this type are generally sold as disposable items and are intended to be used only once to negate the risk of transmission of diseases between patients. However, such syringes suffer from the drawback that it is difficult to prevent such syringes from being re-used, which re-use increases the risk of transmission of serious, life-threatening, conditions such as certain bacterial infections, viral hepatitis, and HIV.

Numerous designs have been proposed for syringes which are intended to negate or reduce the risk of the syringe being re-used. However, there are considerable challenges involved in designing a syringe which meets all desiderata, including, without limitation:

(a) allowing aspiration or flashback of blood to check for correct location of the needle in a patient;

(b) ability to deliver variable doses;

(c) smooth operation;

(d) simplicity of manufacture and use

(e) ability to inject diluent (e.g. sterile water) into a vial of powdered/lyophilised drug, and/or allow agitation of vial or syringe contents to assist powdered drug to go into solution.

EP0925083B1 discloses a single use syringe comprising a barrel with an internal annular groove at the proximal end and a further annular groove near the distal end but spaced from it. The plunger is formed with an integrally moulded barb-like flange adjacent the head, which flange bears resiliently against the barrel interior wall. The flange is able to move unrestricted in either proximal or distal direction along the majority of the barrel interior wall; however, the flange is only able to pass the annular grooves in the distal direction. The syringe is supplied with the plunger not fully depressed, so that the restrictor flange is on the proximal side of the more distal of the two grooves. Liquid may be drawn up into the syringe until the flange encounters the proximal groove, which prevents the plunger being withdrawn completely from the barrel. Liquid may be discharged freely from the syringe by depressing the plunger, the flange passing just beyond the more distal of the grooves when the plunger head is moved to the extreme distal end of the barrel. In this position, the plunger is now prevented from being withdrawn again because the flange will not pass the groove in the barrel. This design is elegant and simple but suffers from a number of drawbacks, not least the fact that there will inevitably be a slight jolt as the flange passes the more distal of the grooves when an injection is being given, and this jolt is likely to be felt by a patient. Furthermore, because of the unrestricted movement of the plunger between the two grooves, the syringe could potentially be used again and again provided the plunger is never fully depressed. After unpacking a syringe, it is normal to cycle the plunger over a short distance to check that the plunger is free to move and, if it is not, to free it: sometimes there can be a degree of adhesion between the plunger head and the barrel due to the length of time of storage, or due to the effects of gamma sterilisation. This is particularly the case with plunger heads which have had silicone lubricant applied to them. During this movement it would be relatively easy to lock the plunger of this syringe by moving the flange past the distal groove. This design is the only one of which the inventors are currently aware which properly can be used to aspirate a flash of blood for checking needle position in a patient prior to injecting a drug.

U.S. Pat. No. 5,000,737 discloses a syringe having a single piece metal barbed restrictor element located between the plunger shaft and the cylindrical interior syringe barrel wall. The element has barbs facing towards the plunger which prevent movement of the plunger distally with respect to the element, and barbs facing the barrel which prevent movement of the element proximally with respect to the barrel. The restrictor element is initially located near the proximal end of the barrel; thus initial proximal movement of the plunger to draw up liquid is permitted as the plunger can slide past the restrictor in this direction. Subsequent depression of the plunger to deliver liquid is permitted because the restrictor can move distally with respect to the barrel, and hence when the plunger is depressed it carries the element with it. Further movement is of course prevented. This design has many similarities with some of the embodiments set out below; however, it does not permit aspiration of a flash of blood nor repeated movement to assist in reconstitution of lyophilised drug.

US2003/0060759 discloses a design which has similarities to that of U.S. Pat. No. 5,000,737, but also some important differences. It, too, utilises a single piece metal barbed restrictor element mounted between the plunger shaft and the barrel interior wall, and it employees outwardly facing barbs to restrict the motion of the element with respect to the barrel. In this design, however, the plunger shaft has a stepped form with a shoulder part way along it. The restrictor element has a spring tang which acts against the barrel and forces it against the plunger shaft. The restrictor starts out at the proximal end of the shaft; withdrawal of the plunger past the restrictor is permitted until an enlarged diameter portion of the shaft, near the plunger head, comes into engagement with the restrictor. At the same time, the proximal end of the restrictor snaps behind the shoulder on the plunger; thereby movement of the plunger in either direction with respect to the restrictor is prohibited. The plunger can be depressed, carrying the restrictor with it to the distal end of the barrel, and then the plunger is incapable of further movement. This design is simple and has been used in a commercial vaccination syringe product. It suffers from the disadvantage that it may be used repeatedly, provided the user does not draw up the plunger to the point where the restrictor snaps into place on the reduced diameter part of the plunger shaft. Furthermore, once the restrictor has locked into place, which is of course the intention, aspiration of a flash of blood is not possible.

U.S. Pat. No. 5,222,942 discloses a design based on a ratchet system. A collar is installed in an initial distal position between plunger shaft and barrel. The plunger shaft is formed with annular ratchet teeth, and corresponding teeth are formed on the collar. The ratchet does not permit the plunger to be moved proximally past the collar, so when the plunger is initially drawn back in order to draw up liquid, it carries the collar with it to the extreme proximal end of the barrel where a formation on the barrel prevents the collar and plunger from being withdrawn completely from the barrel. The ratchet is such that the plunger may then be depressed past the collar to dispense liquid, and then of course the syringe is disabled. This design does not allow for aspiration of a flash of blood.

### DEFINITIONS

Throughout this specification, the terms “distal” and “proximal” will be interpreted with respect to the user of the syringe, i.e. the person administering an injection. Thus the “proximal” end of the syringe is the open end into which the plunger is received, and the “distal” end is the nozzle/needle end.

The terms “usable length” and “usable extent” as used herein with respect to a syringe barrel means that portion of the barrel's length over which the plunger head is intended to be able to travel in the course of normal use, that is to say in the course of drawing up and discharging/injecting fluid. In some cases this can be a relatively small proportion of the overall length of the syringe, e.g. if it is desired that a restrictor bobbin be inserted deep into the barrel so as to make it harder for it to be removed by a user who may wish to deactivate the single use feature of the syringe.

The term “movement” as used herein, unless stated to the contrary, refers to movement substantially along the axis of the syringe, that is to say along the length of the syringe. Similarly the term “direction”, as used herein with regard to the movement of components, refers to one or the other direction along the axis of the syringe, i.e. the proximal sense or the distal sense.

The terms “restricted” and “restrict” as used herein with respect to movement of a component of the syringe with respect to another component are intended to mean that a degree of restriction of movement is provided which is appropriate for the particular syringe. What is important is that the overall design of the syringe is such that a user attempting to circumvent its non-reuse features is prevented from doing so or is at least severely hampered. Different degrees of “restriction” may be required for different designs. In modified versions of some of the embodiments described herein, the plunger may have a weak point and be designed to break if a user tries to move the plunger in a restricted direction, thereby rendering the syringe inoperable. In a syringe incorporating such a feature, the force needed to move syringe components in a “restricted” direction may not be very great, e.g. 30-100N, provided the plunger is designed to break when a force lower than this is applied. A syringe in which the plunger did not have such a weak point may require that a greater force is able to be resisted.

### SUMMARY OF THE INVENTION

According to a first aspect of the present invention, a syringe comprises:

(a) a barrel having a cylindrical interior surface substantially free of discontinuities over its usable extent;

(b) a plunger including a plunger head and a shaft;

(c) a restrictor bobbin movable with respect both to the barrel and the shaft to permit the drawing up and delivery of a fluid over the usable extent of the barrel, whilst limiting further use of the syringe;

(d) the restrictor bobbin and/or shaft together comprising one or more members which are freely movable with respect to each other over a limited distance which is smaller that the usable extent of the barrel so as to permit repeated distal and proximal cycles of movement of the plunger head over the said limited distance.

A syringe barrel which is substantially free of discontinuities over its usable extent helps to make for smooth operation of the plunger, especially during the delivery stroke when an injection is being given to a patient. Any jolts in the operation of the syringe during the delivery stroke are normally felt by the patient, and it is desirable to avoid this happening. Discontinuities in the inner cylindrical surface which would not be engaged by the sealing face of the plunger head (piston) in normal use or in use during the delivery stroke are not considered to be in the “usable extent” of the barrel. For example, a reduced diameter portion of the barrel may be provided at the far proximal end of the usable extent of the barrel for preventing complete withdrawal of the plunger from the barrel: this would not be considered part of the “usable extent”.

The provision of free relative movement of the restrictor bobbin and shaft over a limited predetermined extent allows repeated movement over a distance which is sufficiently small not to allow repeated injections with the syringe, or at least only to allow repeated injections of smaller quantities of fluid than the syringe would normally be capable of delivering. This degree of movement may be sufficient to allow for aspiration/flash-back of blood for checking needle location in a patient. Alternatively or in addition, this degree of movement may be sufficient for repeated movements to assist reconstitution of powdered/lyophilised drug into solution.

The said member(s) which are freely movable to permit repeated movement of the plunger head may be the restrictor bobbin in its entirety, or a part of it, or alternatively a part of the plunger shaft.

Optionally, the said movable member(s) may be provided by deformable portions of the bobbin or shaft, which are preferably resiliently deformable.

Preferably, said repeated movement is permitted at least when the plunger head is at or adjacent a proximal end of the said usable extent of the barrel.

It is preferable, though not essential, that the feature which allows aspiration of blood be operative at any position of the plunger so that the feature can be used whatever volume of injectable is contained in the syringe and also may be used to check the needle position in a patient prior to drawing a blood sample, i.e. when the syringe is substantially empty and the plunger in a distal position with respect to the barrel. Therefore, preferably the said limited repeated cycles of distal and proximal movement referred to above are permitted at substantially every relative position of the plunger and barrel over a the usable range. Furthermore, desirably the resistance to movement offered by the said freely movable members is such that proximally directed force on the plunger will move the said members in preference to any other means for moving the plunger with respect to the barrel. Repeated movements of the plunger over a small distance is thereby possible when the plunger is not at its most proximal position without inadvertently effecting further “permanent” retraction of the plunger.

The said free movement of the bobbin and shaft with respect to each other may be permitted in a first region of the shaft and resisted in a second region of the shaft.

The distance over which repeated movement is possible is, desirably, sufficient to aspirate a small volume of blood from a patient so as to check the position of the needle. All that is required for this function normally is that the aspirated blood be visible in the syringe. Factors which may need to be taken into account in determining the degree of movement required for this function may include, without limitation:

(a) the internal volume of the needle;

(b) if the needle is a separate entity, the internal volume of the syringe nozzle onto which the needle hub fits and any volume between the end of the nozzle and the internal base of the needle hub;

(c) any volume between the internal end of the syringe barrel and the plunger face when fully depressed;

(d) any “end float” of the plunger head: if the plunger head is a separate entity from the shaft, then a certain amount of free play between the two is sometimes required to ensure the head “snaps on” to the shaft in manufacture;

(e) resilient deformation of the plunger head;

(f) the pressure drop which it is necessary to create to be sure that blood is aspirated;

(g) the volume of blood which needs to be present in the syringe barrel for the user to be able to discern its presence;

(h) a safety/error factor;

(i) the distance over which the user can discern movement easily: the “ergonomics” of the feature; and

(j) the diameter of the syringe barrel and plunger head.

The last of these points particularly will have a large effect on the volume as swept by the plunger head which corresponds to the distance over which the said repeated movement is possible.

The smallest possible volume for achieving this effect is about 10 microlitres: this might be the case e.g. if a short and thin (e.g. 1 cm, 30 gauge) needle is used which is moulded into the syringe barrel and if a plunger with an integrally moulded head is used, etc. In a 10 ml syringe this would correspond to 0.1% of the 10 ml swept volume of the syringe which equates to 0.1% of the usable length of the syringe. This is not precise since the usable volume of a syringe is often slightly more than its stated or graduated volume.

Normally a volume considerably greater than 10 microlitres would be required. For example, a large needle (e.g. 5 cm long, 18 gauge) may have an internal volume of approximately 50 microlitres. A standard luer nozzle has a dead space of about 50 microlitres and there will be additional dead space between the end of the nozzle and internal end of the needle hub. The plunger head of some syringes may have an end float of up to 1 mm which could correspond in a 10 or 20 ml syringe to 500 microlitres or more. In a 10 or 20 ml syringe a very small volume of blood may be more difficult to see in which case as much as 500 microlitres may be required. Adding these factors together with an allowance for error and for creating the pressure drop for withdrawing the blood might give a volume of as much as 2000 microlitres. In a 20 ml syringe this would correspond to 10% of the usable (graduated) swept volume of the syringe.

If it is desired that the syringe plunger should be capable of repeated movement of sufficient extent to agitate a drug powder and diluent mix, then it may be desirable to increase this range to as much as 50%. Of course, the larger the range of free movement, the greater the danger that this movement makes the syringe too easily re-usable for injecting drugs or other uses. The figure of 50% would probably be much too high for a 10 or 20 ml syringe, but for a very small syringe (0.5 ml or less) it may not be totally unreasonable from the point of view of preventing or at least hindering further use.

It can be seen that a large range of possibilities exist depending on the exact use to which the syringe is to be put. However, for most situations, a volume of between 50 and 500 microlitres would be preferable. 50 microlitres might be appropriate e.g. for a 5 ml syringe with a very small integral needle and where agitation of reconstituted drugs is not required. It would probably be desirable for this quantity to be more in the region of 100 or 150 or 200 microlitres, however, to allow a good margin for safety, human error, manufacturing tolerance, etc. 1000 microlitres might be appropriate for the same syringe where the ability to agitate reconstituted drugs is desirable, though this may still provide too great an opportunity for re-use of the syringe, and 500 microlitres may be more preferable.

Preferably, the distance over which the said repeated movement is possible is between 0.1% and 50% of the said barrel usable length, preferably between 1% and 20%. The lower end of this range might preferably be increased to 2%, 3% or 4% based on the example discussed above. The upper end of this range might preferably be reduced to 10% based on the example discussed above. However, these ranges should not be taken as limited to the particular syringe sizes discussed above which are presented by way of example only.

Accordingly, the said distance over which repeated movement is possible corresponds to a swept volume which is between 10 and 2,000 microlitres, preferably between 50 and 1000 microlitres, more preferably between 100 and 500 microlitres, or other absolute volume ranges corresponding to the percentage values of the total syringe volume mentioned above, for syringe total usable/measurable volumes of 0.5 ml, 1 ml, 1.5 ml, 2 ml, 2.5 ml, 3 ml, 5 ml, 10 ml and 20 ml.

The penultimate item in the above list of factors which may affect the desirable range of repeatable movement, factor (i), may require that a minimum distance is determined by what a user can practically work with. A reasonable range might be 0.5 mm to 20 mm, preferably 1 mm to 15 mm, more preferably 1.5 mm to 10 mm, still more preferably 2 mm to 7 mm or about 3 mm or 4 mm.

One or more of the said movable or deformable members which allow repeated movement of limited extent may incorporate or comprise a projection, tine, tang, barb, serration or other like formation or member in engagement with the barrel interior wall or the plunger to restrict motion of the said member(s) with respect to the barrel interior wall or plunger respectively in a predetermined direction (either proximal or distal).

The plunger may be provided with formations for restricting the movement of the restrictor bobbin with respect to the plunger shaft. One possibility is for the plunger shaft to be provided with a region, preferably defined by stop surfaces at each end of the said region which the bobbin cannot or cannot easily pass, over which region unrestricted repeated movement of the shaft with respect to the restrictor bobbin is possible.

The restrictor bobbin may take the form of a unitary member, which may have no slidable or substantially deformable parts, e.g. may be a unitary metal (e.g. pressed stainless steel) component. The bobbin may have one or more tines, barbs, serrations or the like formation(s) in engagement with the barrel interior wall so as to prevent movement of the bobbin with respect to the barrel in a predetermined direction, preferably whatever the position of the bobbin over the usable length of the barrel.

In addition to a first region as described above over which unrestricted movement of the shaft with respect to the bobbin is possible, the plunger shaft is preferably provided with a second region over which movement of the shaft with respect to the bobbin is restricted in one direction. The second region may be provided, for example, with a ratchet formation in which case the bobbin would be provided with a corresponding formation for engaging the ratchet formation on the plunger shaft, the formations acting together to restrict movement of the plunger with respect to the bobbin in one direction. Alternatively, the second region may be substantially smooth and a barb, serration, tine or similar may be provided on the bobbin facing the plunger and arranged to engage with the second region of the plunger so as to resist movement of the plunger with respect to the bobbin in a predetermined direction, but to permit proximal and distal movement of the plunger with respect to the bobbin over the said first region. The bobbin is preferably provided with a spring member acting against the barrel interior to bias the bobbin into engagement with the shaft at least when the bobbin is in registry with the said second region of the shaft.

The syringe would normally be supplied sterile packed with the plunger fully depressed and the bobbin in its starting position. One possibility if for the syringe to be provided pre-filled with diluent. In this event, a distal movement of the plunger is normally required to expel the diluent prior to a proximal movement to draw up reconstituted drug solution and the a further distal movement to deliver the drug solution. For this situation, it may be desirable to provide a restrictor member on the bobbin whose sense can be reversed, i.e. the direction in which it resists movement may be reversed. This could be achieved using barbs, tines or similar whose direction may be changed e.g. by applying a force to them via the plunger in the distal (depressed) position of the plunger in the barrel.

The syringe is preferably sterile packed with the plunger in a substantially fully depressed position.

Optionally, the syringe may be sterile packed with the plunger in a retracted position, especially if the syringe is supplied ready filled with sterile water or other diluent for reconstituting powdered or lyophilised drugs. In this case, it will be understood that a distal movement of the plunger is required to expel the diluent into a drug vial prior to withdrawing the plunger to draw up the dissolved drug and then expelling the drug solution.

In order to allow this, the invention encompasses the possibility that the bobbin may incorporate a restrictor member for restricting movement of the plunger with respect to the bobbin or the bobbin with respect to the barrel where the direction in which such movement is restricted is reversible.

The restrictor member in this case may comprise barbs or tines whose direction may be reversed by depressing the plunger when it is at or near its most distal position in the barrel.

Second, third and fourth aspects of the invention are set out below. The above preferable or optional features of the first aspect of the invention apply equally to the second, third and fourth aspects of the invention defined below. The above discussion of the range of possible distances and volumes which apply to the limited repeated cycling of the syringe plunger also applies equally to the second, third and fourth aspects.

According to a second aspect of the present invention, a syringe comprises:
(a) a plunger including a plunger head and a shaft;
(b) a barrel having a cylindrical interior surface substantially free of discontinuities over the usable range of movement of the plunger head;
(c) a restrictor bobbin adapted for unidirectional movement with respect to the barrel in a first direction and to the shaft in a second direction to permit the drawing up and delivery of a fluid over the said usable range, whilst limiting further use of the syringe;
(d) the restrictor bobbin and shaft or a part or parts of the bobbin or shaft being relatively movable freely over a predetermined limited distance, at least when the plunger is in a region adjacent the proximal end of the said usable range of movement, so as to permit repeated distal and proximal movement cycles of the plunger head over the said limited distance, the said limited distance being less than the said usable range of movement.

Preferably the said repeated distal and proximal movement cycles referred to above are permitted at substantially every relative position of the plunger and barrel over the said usable range.

Optionally, if a part of the restrictor bobbin or shaft is relatively movable (i.e movable with respect to another part of the bobbin or shaft respectively), then this part may be provided by or may include a deformable member, preferably a resiliently deformable member.

According to a third aspect of the present invention, a syringe comprises:
(a) a plunger including a plunger head and a shaft;
(b) a barrel having a cylindrical interior surface substantially free of discontinuities over the usable range of movement of the plunger head;
(c) a restrictor bobbin interengagable with and movable with respect to both the plunger and the shaft so as to restrict repeated cycles of distal and proximal movement of the plunger head other than over a predetermined limited range, the said predetermined limited range of repeated movement being available at least when the plunger is at or adjacent a proximal end of the said usable range of movement.

According to a fourth aspect of the present invention, a syringe comprises:
(a) a plunger including a plunger head and a shaft;
(b) a barrel having a cylindrical interior surface substantially free of discontinuities between proximal and distal ends of a full range of usable movement of the plunger head in the barrel;
(c) a restrictor bobbin located between the shaft and the barrel and having outer barbs, tines, serrations or the like interengagable with the barrel to restrict substantial movement of the said barbs, tines, serrations or the like with respect to the barrel in a predetermined direction;
(d) the restrictor bobbin and/or shaft carrying formations for limiting movement of the bobbin with respect to the plunger;
(e) the plunger and bobbin being freely slidable with respect to each other over a limited range of movement, or the plunger or bobbin having relatively movable parts, whereby repeated proximal and distal cycles of plunger head movement with respect to the barrel are permitted over a predetermined limited range which is less than the said full usable range of movement of the plunger head;
(e) the said repeated cycles of movement being permitted at least when the plunger is at or adjacent the said proximal end of the of the usable range of movement of the plunger head.

According to a fifth aspect of the present invention, there is provided a syringe comprising:—

a barrel for containing fluid and having at least one aperture adjacent a first end thereof;

a piston having at least one shaft extending therefrom and adapted to be displaced in said barrel in a first direction from a first position to a second position to cause fluid to enter the barrel through at least one said aperture, and in a second direction from said second position to a third position to cause fluid to be expelled through at least one said aperture;

at least one restrictor bobbin mounted between said at least one shaft and said barrel for sliding movement relative to said barrel and the corresponding shaft;

at least one first gripping member acting between a respective said restrictor bobbin and a respective said shaft for sliding movement relative to said shaft, wherein at least one said first gripping member has a greater resistance to sliding movement relative to the corresponding shaft in said first direction than in said second direction, such that movement of said piston from said second position to said third position causes at least one said restrictor bobbin to move along said barrel in said second direction; and

at least one second gripping member acting between a respective said restrictor bobbin and said barrel to cause the corresponding said restrictor bobbin to have a greater resistance to sliding movement relative to the barrel in said first direction than in said second direction, such that movement of the piston from said third position to said second position, subsequently to movement of said piston from said second position to said third position, without damaging said syringe, is prevented.

By providing a syringe in which movement of the piston from said third position to said second position subsequently to movement of said piston from said second position to said third position is prevented, this provides the advantage of preventing refilling of the syringe after the piston has been displaced from the second position to the third position to expel fluid out of the barrel. In other words, the syringe is prevented from being re-used after it has been used to administer an injection or remove bodily fluids, as a result of which the risk of transmission of disease is significantly reduced.

In a preferred embodiment, the first position is substantially coincident with the third position.

At least one said second gripping device may be adapted to engage said barrel such that movement of the corresponding said restrictor bobbin in said first direction relative to the barrel causes damage to the surface of said barrel to prevent said piston subsequently forming a fluid seal with said barrel.

This provides the advantage that forced withdrawal of the piston subsequently to movement of the piston from the second to the third position causes damage to the smooth walls of the barrel, thereby destroying the integrity of the fluid seal between the piston and the barrel. As a result, the syringe can no longer generate the necessary suction to be filled with fluid or pressure to expel fluid. This in turn makes re-use of the syringe more difficult.

At least one said first gripping member and the remaining part of the restrictor bobbin may be adapted to cooperate to allow limited sliding movement of said piston relative to the barrel in said first direction subsequently to movement of said piston from said second position to said third position.

This provides the advantage of enabling slight withdrawal of the piston during use to determine whether a needle connected to the syringe has been inserted into a blood vessel. For example, an intramuscular injection, to be injected into muscle tissue, should not be injected into a blood vessel, and slight withdrawal of the piston causes a visible amount of blood to be drawn into the barrel if the needle of the syringe has punctured a blood vessel. However, this safety feature will not permit a significant amount of injectable material to be subsequently withdrawn into the syringe after the primary injection has occurred. The discussion above regarding the degree to which this slight withdrawal is permitted, in terms of distance of movement of the plunger or aspirated volume, applies here.

At least one first gripping member and the corresponding restrictor bobbin may surround the corresponding shaft, wherein the gripping member is adapted to move relative to the corresponding restrictor bobbin between first and second stop positions.

At least one said first and/or second gripping member may comprise at least one metal tine.

At least one first and/or second gripping member may comprise elastomeric material.

An inner wall of the barrel may comprise a first plastics material, and at least one second gripping member may comprise a second plastics material harder than said first material.

An outer wall of at least one said shaft may comprise a third plastics material, and at least one corresponding said first gripping member may comprise a fourth plastics material harder than said third material.

A number of embodiments of the invention will now be described, by way of example only and not in any limitative sense, with reference to the accompanying drawings, in which:—

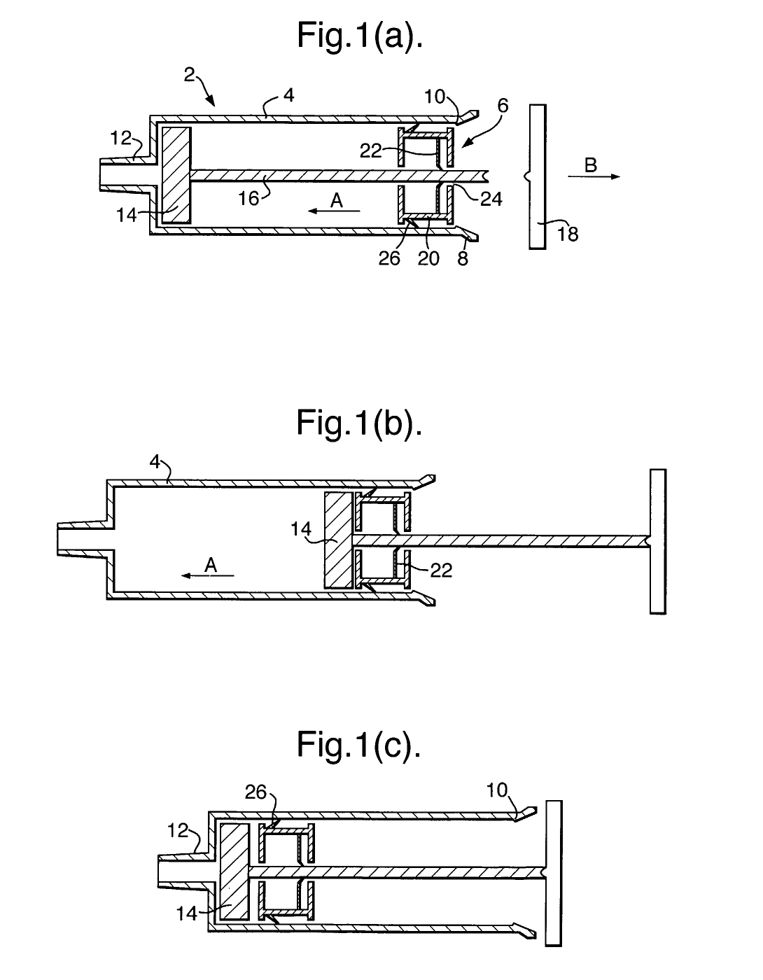

FIG. 1a is a schematic cross-sectional side view of a first embodiment of syringe according to the present invention in an initial manufacturer's packaging position;

FIG. 1b is a view, corresponding to FIG. 1a, of the syringe of FIG. 1a with the piston thereof withdrawn to enable filling of the barrel;

FIG. 1c is a view, corresponding to FIG. 1a, of the syringe of FIG. 1a with the piston thereof depressed to eject liquid from the barrel;

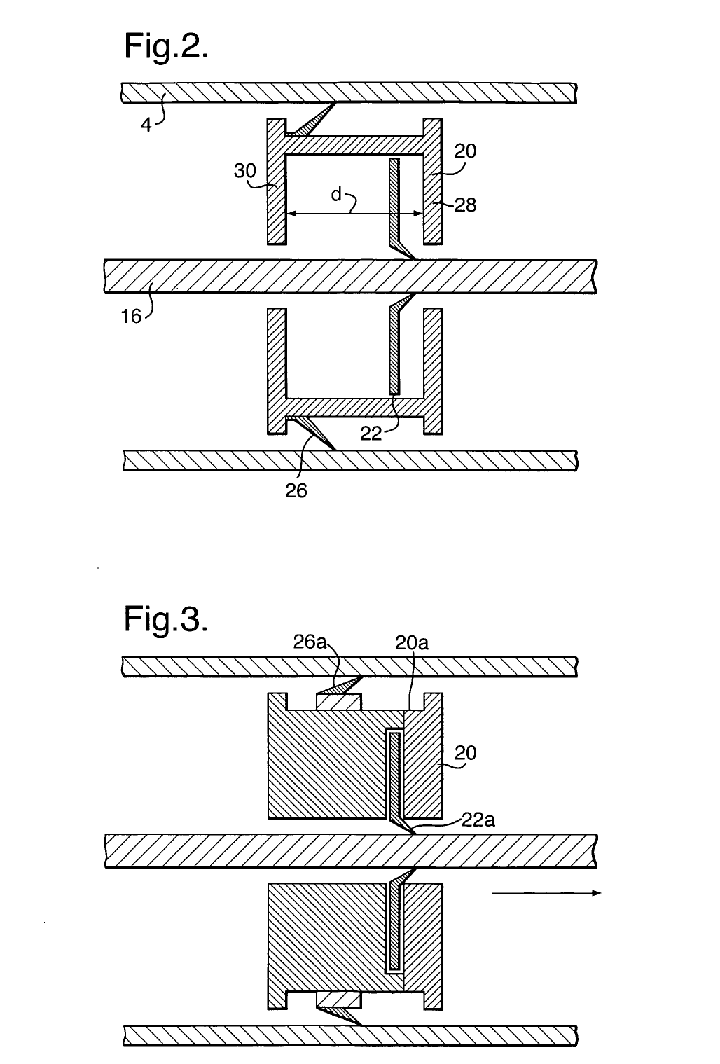

FIG. 2 is a schematic cross-sectional side view of the restrictor bobbin of the syringe of FIG. 1 in the positions shown in FIGS. 1a and 1b;

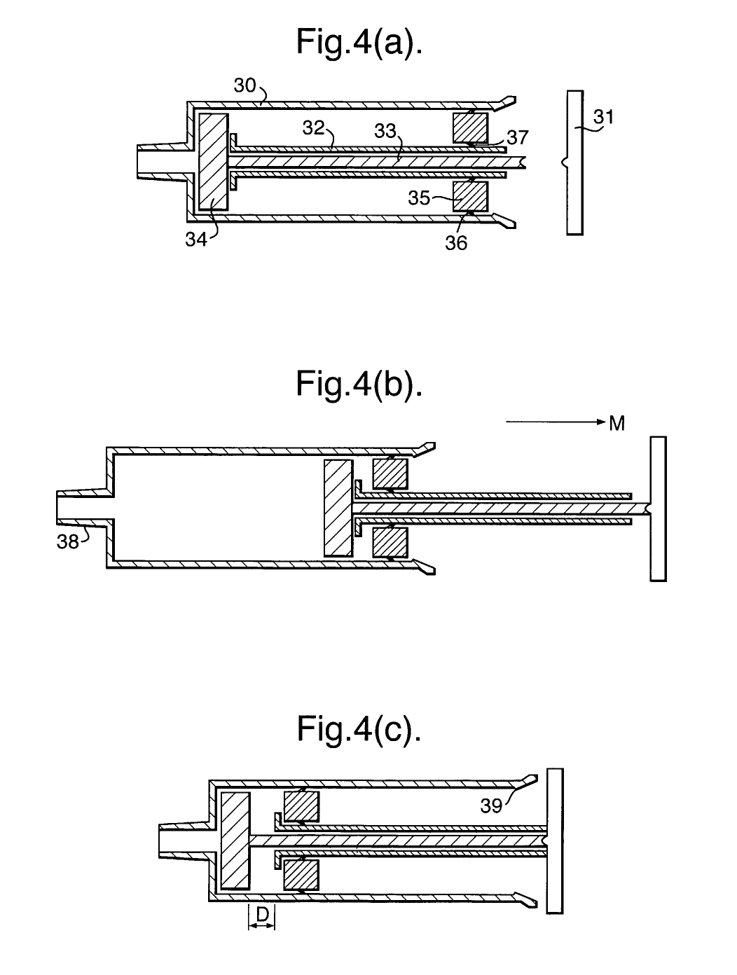

FIG. 3 is a view similar to FIG. 2 of the restrictor bobbin of a modified version of the first embodiment;

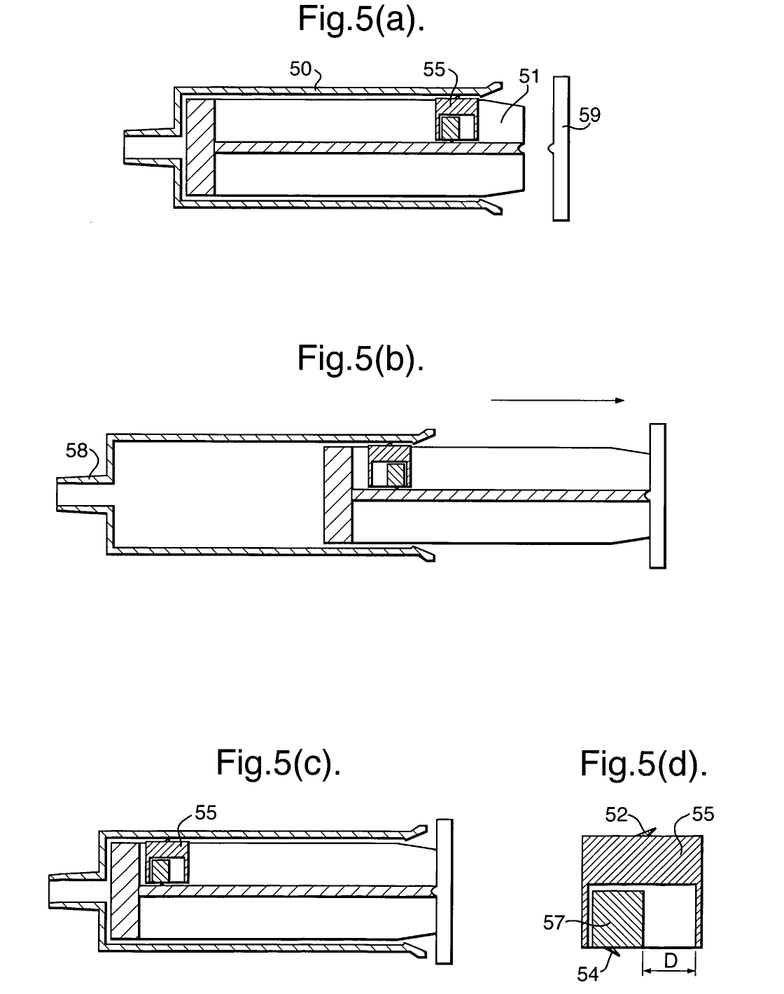

FIG. 4a is a schematic cross-sectional side view of a second embodiment of syringe according to the present invention in an initial manufacturer's packaging position;

FIG. 4b is a view, corresponding to FIG. 4a, of the syringe of FIG. 4a with the piston thereof withdrawn to enable filling of the barrel;

FIG. 4c is a view, corresponding to FIG. 4a, of the syringe of FIG. 4a with the piston thereof depressed to eject liquid from the barrel;

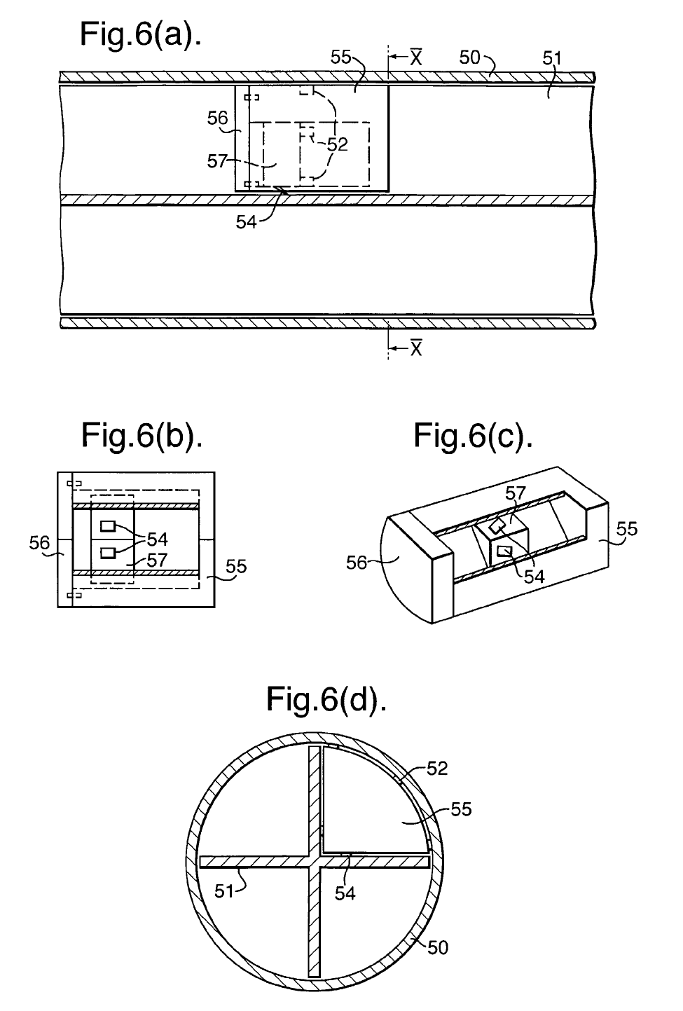

FIG. 5a is a schematic and partly exploded cross-sectional side view of a third embodiment of syringe according to the present invention in an initial manufacturer's packaging position;

FIG. 5b is a view, approximately corresponding to FIG. 5a, of the syringe of FIG. 5a with the piston thereof withdrawn to enable filling of the barrel;

FIG. 5c is a view, approximately corresponding to FIG. 5a, of the syringe of FIG. 5a with the piston thereof depressed to eject liquid from the barrel;

FIG. 5d is a schematic side cross-section of the restrictor bobbin of the third embodiment;

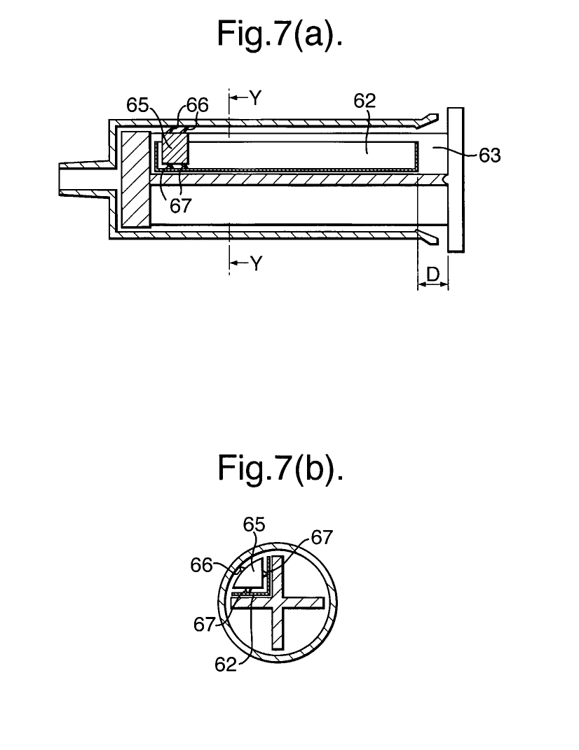

FIG. 6a is a schematic scrap sectional view from the side of part of the third embodiment;

FIG. 6b is a schematic side view of the restrictor bobbin of the third embodiment;

FIG. 6c is a schematic perspective view of the restrictor bobbin of the third embodiment;

FIG. 6d is a schematic sectional view taken on the line X-X in FIG. 6a;

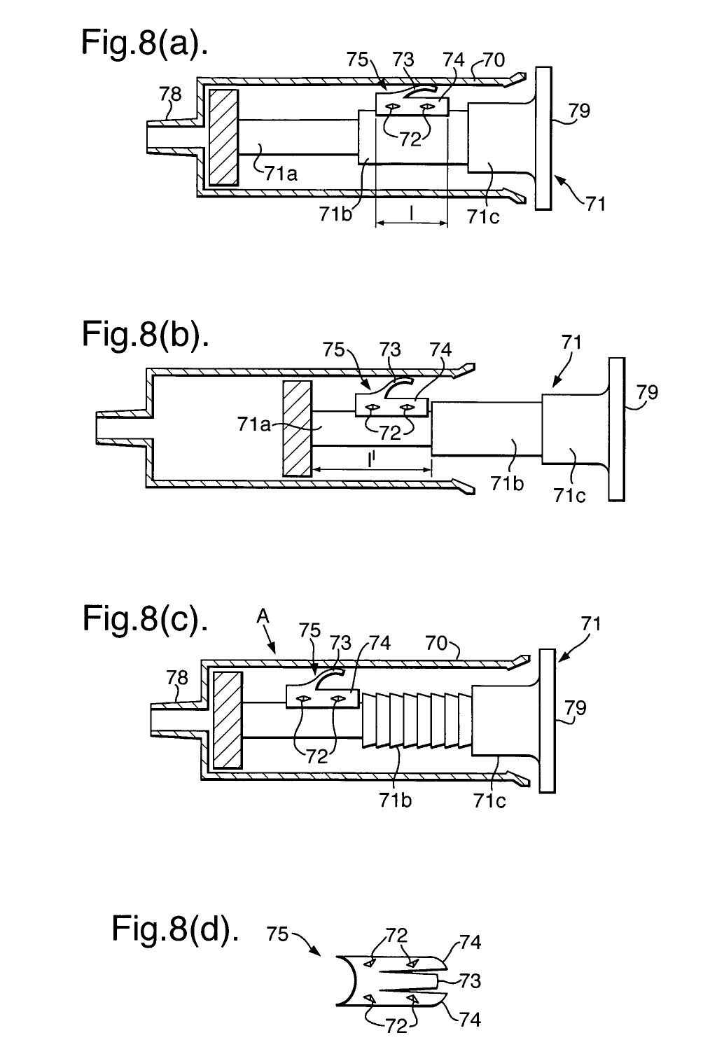

FIG. 7a is a schematic cross-sectional side view of a fourth embodiment of syringe according to the present invention in an initial manufacturer's packaging position;

FIG. 7b is a view, corresponding to FIG. 7a, of the syringe of FIG. 7a with the piston thereof withdrawn to enable filling of the barrel;

FIG. 7c is a view, corresponding to FIG. 7a, of the syringe of FIG. 7a with the piston thereof depressed to eject liquid from the barrel;

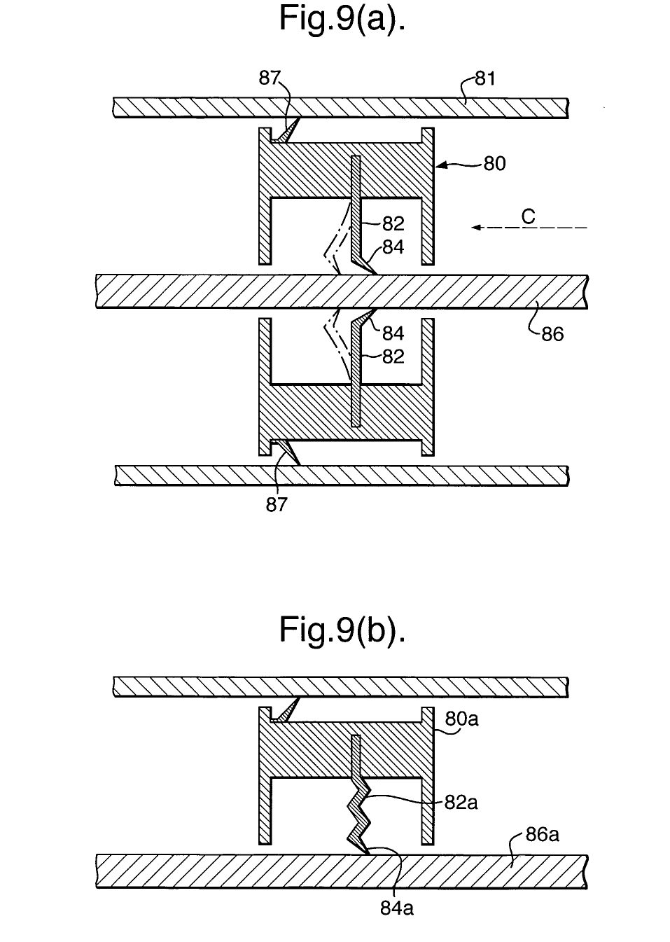

FIG. 8a is a schematic cross-sectional side view of a fifth embodiment of syringe according to the present invention in an initial manufacturer's packaging position;

FIG. 8b is a view, corresponding to FIG. 8a, of the syringe of FIG. 8a with the piston thereof withdrawn to enable filling of the barrel;

FIG. 8c is a view, corresponding to FIG. 8a, of the syringe of FIG. 8a with the piston thereof depressed to eject liquid from the barrel;

FIG. 8d is a view in the direction A in FIG. 8c of the restrictor bobbin of the fifth embodiment;

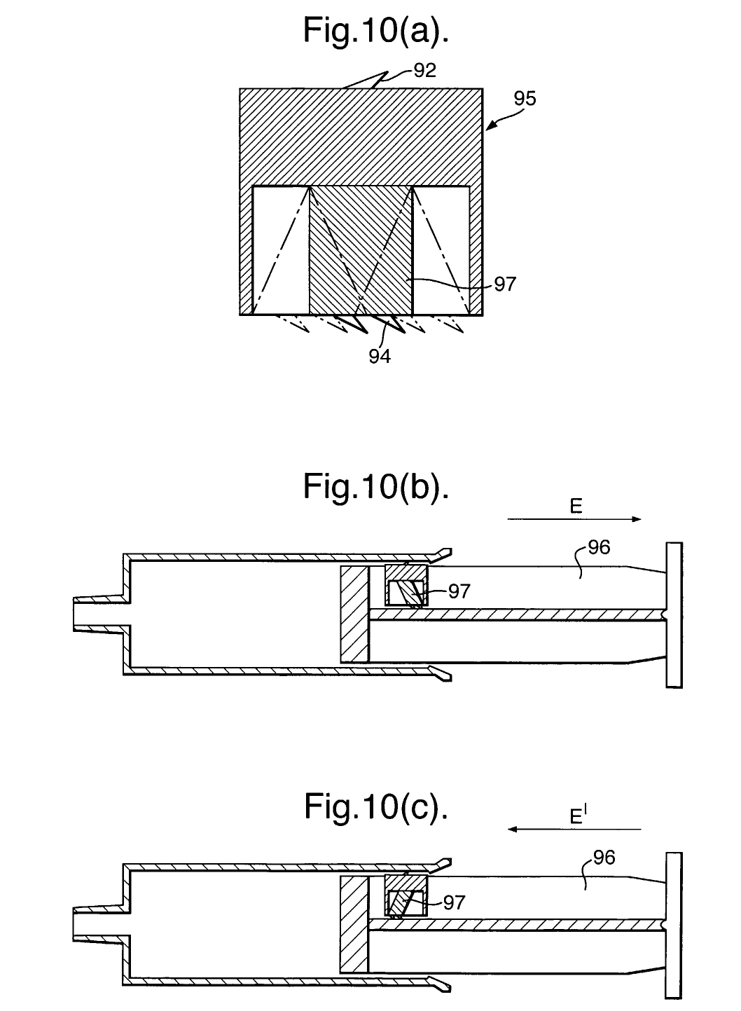

FIG. 9a is a scrap sectional view similar to FIG. 2 of a sixth embodiment;

FIG. 9b is a view similar to FIG. 9a but of only half the diameter of the syringe, showing a modification of the sixth embodiment;

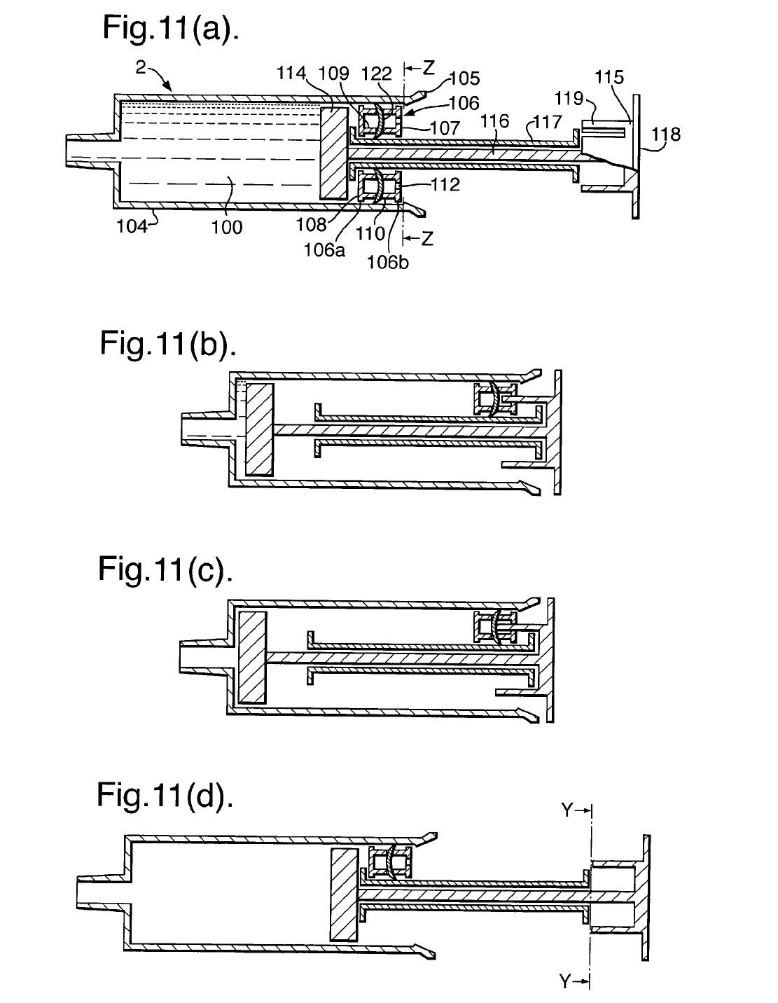

FIG. 10a is a sectional view similar to FIG. 5d of the restrictor bobbin of a seventh embodiment;

FIG. 10b is a side sectional view similar to FIG. 5b of the entirety of the seventh embodiment, showing force being applied to the plunger in a proximal direction;

FIG. 10c is a view similar to FIG. 10b showing force being applied to the plunger in a distal direction;

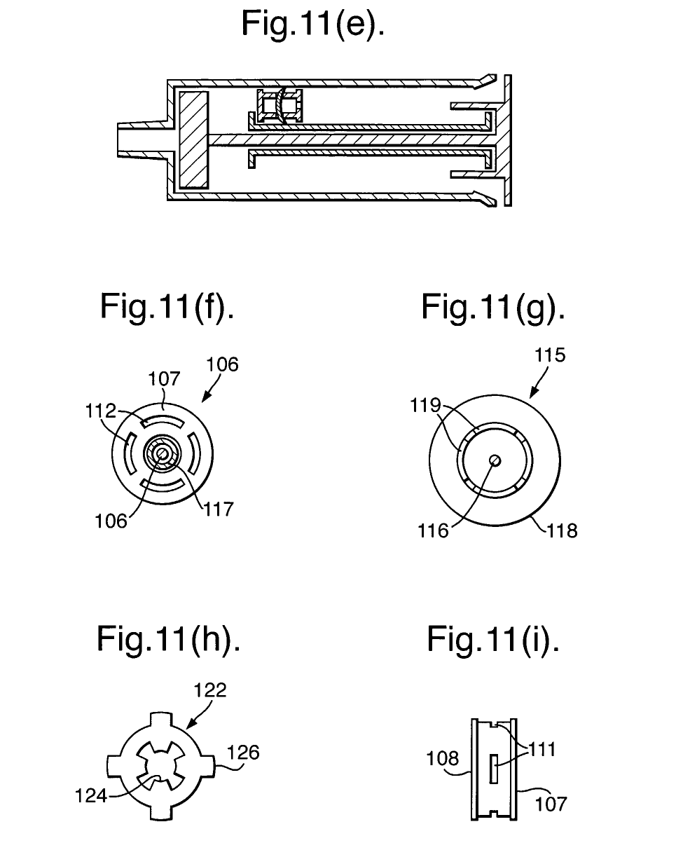

FIG. 11a is a side sectional view of an eighth embodiment as supplied from the manufacturer, charged with diluent liquid;

FIG. 11b is a similar view of the syringe of FIG. 11a, only partly showing the restrictor bobbin, with the plunger almost completely depressed to expel diluent;

FIG. 11c is a similar view to FIG. 11b with the plunger completely depressed to expel diluent;

FIG. 11d is a similar view to FIG. 11b with the plunger retracted having drawn up an injectable liquid;

FIG. 11e is a similar view to FIG. 11b with the plunger depressed after expulsion of injectable liquid;

FIG. 11f is a view along the line Z-Z in FIG. 11a of the plunger shaft (in section) and the restrictor element of the syringe of FIGS. 11a-e;

FIG. 11g is a view along the line Y-Y in FIG. 11d of the proximal end of the plunger of the syringe of FIGS. 11a-e;

FIG. 11h is an axial view of the spring washer shown fully in FIG. 11a and in part in FIGS. 11b-e; and

FIG. 11i is a side view of a part of the restrictor bobbin of the syringe of FIGS. 11a-e.

All the following embodiments are described principally with respect to a syringe having a nozzle, e.g. a luer connector, for attachment of a cannula, hypodermic or other needle or catheter line, etc. It will be appreciated that in every embodiment this nozzle could be replaced by a needle which is incorporated into the syringe at manufacture, e.g. moulded into the plastic of the barrel. It should also be understood that all of the following embodiments may be adapted to provide a frangible or weakened region on the plunger which is designed to break if excessive force is applied to the plunger. Alternatively the plunger may be made in more than one part which parts are designed to separate when excessive force is applied. In either case, the syringe is rendered inoperable or at least substantially inoperable.

Referring to FIGS. 1 and 2, a syringe 2 has a barrel 4 of transparent plastics material having an open end 6 having a widened rim 8 defining an indentation 10 of reduced diameter. The barrel 4 also has an outlet 12 having a needle (not shown) at the end thereof opposite from the open end 6 of the barrel 4.

A piston 14 is slidably received within barrel 4 and has a shaft 16 of plastics material extending from it and having a plunger handle 18 snap-fitted on the end thereof opposite to the piston 14. A safety bobbin or restrictor bobbin 20 of plastics material is slidably received within the barrel 4 and surrounds shaft 16. A gripping washer 22 surrounds the shaft 16 and has tines 24 of metal or plastics material harder than the plastic material of shaft 16 such that the washer 22 grips the shaft 16 and can slide in the direction of arrow A relative to the shaft 16 but cannot slide in the direction of arrow B (FIG. 1a)

A second gripping member in the form of a plurality of tines 26 of metal or harder plastics material than the plastics material of inner wall of barrel 4 surrounds safety bobbin 20 such that tines 26 engage the inner wall of the barrel 4 in a manner such that the safety bobbin 20 can be moved relative to the barrel 4 in the direction of arrow A but cannot be moved in the direction of arrow B. The washer 22 can slide a limited distance d (FIG. 2) in either direction relative to the safety bobbin 20 between end walls 28, 30 of safety bobbin 20.

In order to assemble the syringe 2, the piston 14 together with the shaft 16, with the plunger handle 18 removed from the shaft 16, is inserted into the barrel 4 and pushed along the barrel until it abuts the end of the barrel adjacent to outlet 12. The safety bobbin 20, together with washer 22 and gripping member 26 is then placed around the shaft 16 and snap-fitted into the open end 6 of barrel 4. The safety bobbin 20 is prevented by indentation 10 and gripping member 26 from being removed from the barrel 4. The plunger handle 18 is then snap fit onto the end of shaft 16 remote from piston 14.

The operation of the syringe 2 will now be described.

The syringe 2 is provided by the manufacturer in sterile packaging (not shown) in the condition shown in FIG. 1a but with the plunger handle 18 mounted to the shaft 16. In order to fill the syringe 2, the needle (not shown) extending from outlet 12 is inserted into a reservoir of injectable liquid, or into the body of a patient, as a result of which liquid is drawn into the barrel 4, through outlet 12. The plunger handle 18 is then withdrawn in the direction of arrow B (FIG. 1a) to withdraw the piston 14 until it abuts the safety bobbin 20 as shown in FIG. 1b. In this position, the safety bobbin 20 is captured and locked in the barrel 4, and shaft 16 slides in the direction of arrow B relative to safety bobbin 20 and washer 22 until the piston 14 abuts end wall 30 of safety bobbin 20. At the same time, the washer abuts end wall 28 of safety bobbin 20.

In order to administer an injection, or expel bodily fluid such as blood from the syringe 2, the plunger handle 18 is then pushed in the direction of arrow A, as a result of which the piston 14 moves towards outlet 12 to expel liquid from the outlet 12. At the same time, because of the axial length of safety bobbin 20, rocking of the piston 14 and shaft 16 relative to safety bobbin 20 and barrel 4 is prevented. The washer 22 is prevented from moving in the direction of arrow B relative to shaft 16, but can move distance d relative to safety bobbin 20 until it abuts end wall 30 of safety bobbin 20. Thereafter, as the piston 14 moves in the direction of arrow A and barrel A, the safety bobbin 20 moves along barrel 4 until the piston 14 abuts the end wall of barrel 4 adjacent outlet 12 as shown in FIG. 1c.

If the piston handle 18 is at any point withdrawn in the direction of arrow B, the washer 22 can move distance d within safety bobbin 20 until piston 14 abuts end wall 30 of safety bobbin 20, or the washer 22 abuts the end wall 28 of safety bobbin 20. If the needle extending from outlet 12 has been inserted into a blood vessel, this action will withdraw blood into the barrel 4, which can then be seen by a user. It may also be possible to use this small degree of movement to agitate the drug in the syringe: this may be advantageous e.g. where a powdered drug needs to be agitated in order to ensure that it is fully dissolved into solution in a diluent contained in the syringe, prior to administration of the drug.

At the end of travel of the piston 14 and after the washer 22 abuts end wall 30 (or piston 14 abuts end wall 30) of safety bobbin 20, subsequent movement of the piston 14 in the direction of arrow B is limited only to the small float distance d, large functional movement is prevented by engagement of the gripping member 26 with the internal wall of barrel 4. If sufficient force is applied to the shaft 16 to overcome the resistance of gripping member 26, one or more, or even all, of the tines of gripping member 26 will damage the internal wall of barrel 4 of the syringe 2, resulting in an effective fluid seal between the piston 14 and the inner wall of the barrel 4 no longer being possible. In this way, withdrawal of piston 14 will no longer cause suction in barrel 4, as a result of which the syringe cannot be re-filled with injectable liquid and therefore cannot be re-used.

In a modification of the first embodiment shown in FIG. 3, the sliding washer 22 is replaced by a fixed washer 22a, whilst the sliding function is taken over by the gripping member which is slidably received on the exterior surface 20a of the bobbin 20.

It will be appreciated that the first embodiment, in either of its forms, could easily be adapted so that the direction in which the gripping member and washer resist motion is reversed That is to say, the outer tines or barbs 26 would face distally so as to resist motion of the bobbin in the distal direction with respect to the barrel; the inner tines 24 would also face distally so as to resist motion of the plunger proximally with respect to the bobbin. In this event, the bobbin would be in a distal position in the device as manufactured. When the device is first used and the plunger is withdrawn by a user in order to draw up a fluid, the bobbin would be carried back with the plunger towards the proximal end of the syringe barrel. During the delivery stroke, i.e. movement of the plunger in a distal direction, the bobbin would remain fixed with respect to the barrel, and the plunger would move past the bobbin. This variation may also apply to one or more of the following embodiments.

A second embodiment is shown in FIG. 4. In this embodiment, the syringe consists of a barrel 30 with an open end through which the internal working components can be inserted. The other end is essentially closed, apart from a portal/nozzle 38 adapted for attachment of a needle or catheter as is conventional. The plunger includes a piston/bung or plunger head 34 and a circular cross section shaft. The shaft has two parts: a core 33 which is connected to the head 34 and to a handle 31, and an outer sleeve 32 which is slidable on the core 33.

The outer sleeve 32 is somewhat shorter in length than the plunger core 33. Movement of the sleeve 33 on the core 32 is limited to the distance D shown in FIG. 4c by engagement of the sleeve 32 with the plunger head 34 and the handle 31.

An annular restrictor bobbin 35, having a diameter smaller than the internal diameter of the syringe barrel, is located around the plunger sleeve 32. The central aperture of the restrictor bobbin 35 is slightly larger than the external diameter of the plunger sleeve 32. Affixed to both its outer periphery and to the wall of the central aperture are a number of tines or barbs 36, 37 respectively. The tines are directed in such a way that they will only permit movement of the plunger sleeve in the direction M shown in FIG. 4b.

During withdrawal of the plunger to draw up injectable or diluent, the outer tines 36 engage the interior wall of the syringe barrel and prevent the slidable restrictor bobbin from being displaced. A reduced diameter section 39 of the barrel at its extreme proximal end provides further security against the restrictor bobbin being either inadvertently or deliberately withdrawn from the barrel.

When the user depresses the plunger to expel the syringe contents, the plunger sleeve abuts the handle 31 and both outer shaft and core move distally. The inner tines 37 engage the plunger sleeve 32 and drag the restrictor bobbin 35 down the barrel of the syringe to a position at the extreme distal end of the syringe barrel. The outer tines 36 then prevent the subsequent withdrawal of the plunger to any substantial extent.

At any time during manual operation of the syringe, the plunger shaft core 33 and connected head 34 can be moved independently back and forth a small distance D. This allows for aspiration of a small quantity of blood or for repeated movement to assist in reconstituting a drug prior to injection.

Referring now to FIGS. 5a to 5d and to FIGS. 6a to 6d, a third embodiment is shown which operates on similar principles to the other embodiments but involves a syringe with a cruciform section rather than a circular one.

FIGS. 5a-5c show the third embodiment in (a) its position as supplied from the manufacturer, (b) the drawn back position and (c) the position after expulsion of the contents. These are largely self explanatory in view of the foregoing descriptions of the first and second embodiments.

FIG. 5d shows in somewhat more detail the restrictor bobbin of the third embodiment, which comprises a sector-shaped housing having external tines 52. The housing defines a recess in which is received a sliding element or member 57 which is provided with a further set of tines 54 constituting the internal tines of the overall bobbin. The distance over which repeated movement is provided by the bobbin is marked as D in FIG. 5d.

Further detail of this embodiment is shown in FIGS. 6a to 6d. FIG. 6a shows schematically a cross section of part of a syringe barrel 50 and plunger 51. Received in one quadrant of the cruciform plunger 51 is a slidable restrictor bobbin. As may be seen more clearly with reference to FIGS. 6b-d, the bobbin has a recess along running along the length of its corner edge which sits in the internal corner of the plunger shaft quadrant. Slidably received in this recess is a slider element 57 which is provided with barbs or tines 54 for engaging with the plunger shaft. A removable end cap 56 of the bobbin allows the slider element 57 to be inserted into the recess during manufacture. On the curved outer surface of the bobbin is a further tine or tines 52 which engage the barrel interior wall.

The operation of this embodiment will be easily understood from the description of previous embodiments. The slider element is captive within the bobbin and can move freely over the length of the recess, thus providing for a limited range of repeated movement for aspiration and/or assisting dissolution of powdered drugs. The direction of the respective tines 52, 54 will depend on where the bobbin is to be located in the manufactured syringe, as will be understood from the description of the previous embodiments.

FIGS. 7a and 7b show a fourth embodiment which is similar in most respects to the second embodiment shown in FIGS. 4a-c. The fourth embodiment relates to a syringe with a plunger having a cruciform section, where a slidable sleeve 62 is provided which fits into one quarter of the main cruciform plunger shaft core 63.

Double sets of inner and outer tines 67, 66 are shown; this arrangement may increase the stability of the bobbin 65. This double tine arrangement may be applied to any of the other embodiments for the same reasons.

FIGS. 8 (a)-(d) show a fifth embodiment of the invention. As with previous embodiments, the syringe comprises a barrel 70 and plunger 71, the plunger shaft in this case being of circular cross section and having a stepped profile with regions 71a, 71b and 71c of different diameter. A restrictor bobbin 75 of generally U shaped cross section sits on the plunger shaft: a detailed view of the bobbin is provided in FIG. 8(d) which is an elevation of the bobbin alone in the direction A shown in FIG. 8(c).

The bobbin 75 is of similar design to that described in prior patent application number US2003/0060759, and the contents of this application are incorporated herein by reference. It is provided with a spring leaf 73 and outwardly and proximally oriented barbs 72. When installed in the syringe, the spring leaf bears resiliently against the interior wall of the syringe barrel 70, thereby urging the barbs 72 against an opposing portion of the barrel interior wall. At the same time, the spring leaf acting against the barrel wall urges the bobbin against the plunger 71. The bobbin is also provided with inwardly directed resilient tangs 74 which grip the plunger shaft, whilst allowing distal movement of the shaft with respect to the bobbin.

FIG. 8(a) shows the syringe in its starting position, with the plunger at its most distal position and the restrictor bobbin 75 located towards the proximal end of the barrel 70. The bobbin sits on the centre region 71b of the plunger shaft which is of smaller diameter than the most proximal region 71c but larger diameter than the most distal region 71a.

In use, a needle will be mounted on the luer connector nozzle 79 and an injectable liquid, e.g. a drug, will be drawn up into the syringe. In a modification of this embodiment, a syringe could be pre-fitted to the syringe in manufacture, in which case the luer connector in FIG. 8 would be replaced by a needle moulded into the plastic of the barrel 70.

One the injectable has been drawn up, the plunger will be in the position shown in FIG. 8(b). Whilst the plunger was being drawn back, the bobbin remained stationary since any tendency for the plunger to carry the bobbin along with it would have been resisted by the barbs 72 engaging with the interior wall of the barrel. The bobbin now sits on the smallest diameter region 71a of the shaft, the tangs 74 having snapped inwardly against the smaller region 71a as they passed over the shoulder between the centre region 71b and the distal region 71a.

As can be seen in FIG. 8(b), the bobbin and plunger are free to move relative to one another over a limited distance defined by the clearance between the distal and proximal ends of the bobbin and the plunger head and plunger shoulder respectively. The length of the bobbin is selected so that enough movement is permitted to allow the aspiration of a small quantity or “flash” of blood as previously discussed. Note that it is not possible to retract the plunger further in the proximal direction so as to remove it and thereby remove the restrictor bobbin to allow further use of the syringe.

In FIG. 8(c) is shown the position of the plunger once the injectable has been delivered. The plunger is at the distal end of the barrel, having carried the restrictor bobbin with it: movement of the bobbin in the distal direction with respect to the barrel is of course permitted by the barbs 72. Once in this position, further retraction of the plunger is substantially prevented. Although the small degree of movement of the plunger permitted by relative sliding of plunger and bobbin is still possible, the dimensions of the components are chosen so that this degree of movement is insufficient to allow further injections.

A potential issue with the fifth embodiment is the possibility that the syringe may be used to draw up and deliver a relatively small volume of drug which did not require the plunger to be drawn back all the way to the position shown in FIG. 8(b). If the plunger is drawn back to a position intermediate those shown in FIGS. 8(a) and (b) then the bobbin may not snap into place in the distal region 71a of the plunger. In this case, the syringe could be used repeatedly. In a modification of this embodiment, a ratchet system is provided which operates between the bobbin and the central region 71b of the plunger. This could be provided simply by appropriate corrugations on the surface of the region 71b of the plunger, so that the tangs 74 of the bobbin engage with the corrugations to prevent distal movement of the plunger with respect to the bobbin. In this event, once any degree of proximal movement of the plunger has been made from the position shown in FIG. 8(a), subsequent distal movement will carry the bobbin with the plunger. This is the case until the bobbin snaps into the distal region of the plunger. Whilst in theory a second injection may still be possible, such a system would seriously impede attempts to use the syringe more than once. Corrugations on the central region 71b are shown in FIG. 8(c) only.

A sixth embodiment is shown in FIG. 9a. This embodiment is very similar to the first embodiment described in detail above with respect to FIGS. 1a-c and FIG. 2. Referring to FIG. 9a, a syringe barrel 81 containing a plunger having a shaft 86, is fitted with a restrictor bobbin 80. The outline of the restrictor bobbin 80 is identical with that of the first embodiment and the outer gripping member 87 is identical with that of the first embodiment. The proximal and distal directions in FIG. 9a are reversed from FIG. 2, so proximal is towards the left and distal is towards the right. In this embodiment, the restrictor bobbin will start out in the syringe as supplied from the manufacturer in a distal position in the syringe barrel.

On drawing back the plunger (i.e. moving it to the left in FIG. 9a), the bobbin will be carried back with the plunger shaft 86 since the barbs 84 will be engaged with the plunger shaft. Once a desired quantity of fluid has been drawn up, the syringe needle (not shown) may be inserted into a patient and the plunger withdrawn to check for needle position by attempting to aspirate blood. If the restrictor bobbin has passed to the most proximal position possible for it (limited e.g. by a reduced diameter portion at the extreme proximal end of the syringe), then the free play allowed by the restrictor bobbin becomes important.

Unlike the first embodiment, the inner gripping washer 82 is fixed in the body of the restrictor bobbin 80 and is not slidable with respect to the body of the bobbin 80. Instead the washer 82 is fixedly mounted in the body of the restrictor bobbin 80. It is made of a springy material (stainless steel would be appropriate) and is so dimensioned that it is capable of resilient deformation as shown in dashed lines in FIG. 9a when a moderate pressure is applied to the plunger in the proximal direction C shown in FIG. 9a. It can easily be seen that the resilient deformation of the washer allows repeatable cycles of distal and proximal movement of the plunger with the barbs 84 of the washer 82 remaining in engagement with the plunger. The distance of the repeatable movement will be determined by the geometry of the washer and restrictor bobbin and, to some extent, the modulus of elasticity of the washer 82.

Referring now to FIG. 9b, a modification of the sixth embodiment is shown in which all parts are identical except the washer 82a which has a corrugated configuration. The washer is arranged to be in compression between the bobbin 80a and plunger shaft 86a. The corrugated configuration means that the washer is able more easily to be deformed, and the fact that it is in compression means that, as it deforms, it expands so that the barbs 84a are kept securely in engagement with the plunger 86a.

Referring now to FIG. 10a, a restrictor bobbin 95 of a seventh embodiment is shown, which is similar to the bobbin of the third embodiment (see FIG. 5d). The restrictor bobbin 95 has outward barns 92 for engaging with the barrel and inward barbs 94 for engaging with the plunger. The sliding element 57 of the third embodiment is replaced in the seventh embodiment with a resilient pad 97 e.g. of silicone rubber or other suitable strong elastomer which is securely fixed to the bobbin as shown. Mounted in the pad 97 are the inner barbs 94. In the seventh embodiment, a double set of barbs 94 is provided for increased stability.

Operation of the seventh embodiment will be apparent, but is shown for clarity In FIGS. 10b and 10c. In the extreme proximal position of the plunger 96, it may be desirable repeatedly to move the plunger through proximal and distal cycles of movement e.g. in order to attempt to aspirate blood from a patient. This is permitted by the resilient deformation of the pad 97. Arrows E and E′ in FIGS. 10b and 10c respectively show the direction of force on the plunger 96.

Referring now to FIGS. 11e-h, an eighth embodiment of the invention is a syringe which is pre-filled with diluent and is specifically for use with powdered/lyophilised drugs which require reconstitution by dissolution in a sterile solvent e.g. water.

A large proportion of injectable drugs, especially for use in developing countries, are supplied in powdered or lyophilised form in the vial. Prior to administration, a measured volume of sterile diluent is drawn up into a syringe, and the diluent then introduced into a vial of the drug, e.g. by passing the needle of the syringe through a pierceable septum on the vial. Although the introduction of the diluent, together with subsequent shaking of the vial, may be enough to cause the drug to pass completely into solution, it may also be helpful repeatedly to cycle some or all of the liquid between the syringe and vial.

This process presents two problems to the designer of a non-reusable syringe. The first is that if diluent is to be drawn up and injected into a vial using the same syringe as will be used to administer the drug—which is highly desirable—two complete cycles of proximal and distal movement of the plunger are necessary prior to the syringe being rendered unusable. The second is that repeated cycling of the plunger is desirable to agitate the drug in the syringe and/or vial which is of course completely opposed to the objective of rendering the syringe unusable after a single operation.

A solution to the second of these issues is presented by a syringe which has a range of free repeatable movement which is sufficient to agitate the contents of a vial or to cycle a portion of it between syringe and vial, the range of free movement being sufficiently small that does not provide an opportunity for re-use of the syringe, or at least severely hampers re-use. Any of the previously described embodiments may be provided with a range of repeatable movement which is appropriate for this objective.

The first of the two problems still presents difficulty, however. It is in theory possible to use the small range of repeatable movement to transfer diluent to a vial of powdered drug in a number of small steps, but this may be undesirable because it would add to the total time needed to prepare and administer an injection. The eighth embodiment presents an alternative solution to the problem.

The syringe 102 shown in FIGS. 11a-e is supplied sterile packed in the state shown in FIG. 11a, filled with sterile water 100. The plunger comprises a head 114, circular cross section shaft core 116 and proximal end piece 115. Similar to the second embodiment, the shaft core 116 has received on it an outer shaft sleeve 117 which is slidable with respect to the core 116 over a limited distance determined by the difference in length of the sleeve 117 and core 116. The end piece 115, which will be described in more detail below, is a separate member which is installed on the end of the shaft core 116 during manufacture, after the sleeve 117 and other components have been installed on the shaft. This is not shown in the drawings.

Received onto the sleeve 117 is an annular restrictor bobbin 106 having a central bore and an outer diameter substantially the same as that of the internal diameter of the syringe barrel 104. The bobbin 106 is retained in the barrel by a flange 105 on the end of the barrel 104. The bobbin body is formed from plastics material, e.g. consists of a distal and a proximal moulding 106a, 106b respectively, between which is received a spring washer 122. The two mouldings 106a and 106b are secured together around the washer 122 during manufacture by adhesive, ultrasonic welding or any other suitable technique. When assembled, the bobbin body is an annular member having proximal and distal end faces 107, 108 and concentric annular inner and outer webs 109, 110. The webs 109, 110 each have four rectangular apertures 111 equally spaced around their circumference. In the proximal end face 107 are located four arc-shaped apertures 112.

Captive between the two halves 106a, 106b of the bobbin body is the washer 122. The washer 122 is of springy metal (stainless steel would be suitable) and comprises a ring like member having outer barbs 126 and inner barbs 124. These are best seen in FIG. 11h. The barbs protrude through respective apertures 111 in the webs 108, 109, whilst the main circular portion of the washer remains between the webs. Referring to FIG. 11a, in the device as manufactured the washer has a bowed profile such that both the inner and outer sets of barbs point distally.

The plunger end piece 115 comprises a unitary moulding of a suitable plastics material. It has a conventional end disc 118 at its proximal end and four arc shaped projections 119 extending distally from the end disc 118. The four projections 119 are in registry with the four apertures 112 in the proximal face 107 of the bobbin 106. Formations (not shown) on the plunger shaft 116, core 117 and bobbin 106 prevent relative rotation of these parts to ensure that the projections 119 remain in registry with the apertures 112 in the bobbin end face 107.

The first step in operating the syringe is shown in FIGS. 11b and 11c: the plunger is depressed to discharge the sterile water contents. The operator may choose how much of the water is discharged through a needle into a vial of powdered/lyophilised drug and how much is discarded. FIG. 11b shows the stage immediately prior to the plunger being fully depressed. The bobbin 106 has remained in its starting position in the barrel since the outer barbs 126 are oriented such as to resist distal movement of the bobbin with respect to the barrel 104. The projections 119 on the plunger end piece 105 have entered the apertures 109 in the proximal face 107 of the bobbin so that they touch the washer 122 at the annular apex of its bowed shape. When further pressure is applied to the plunger in the distal direction, the projections force the bowed shape of the washer 122 to flip into the opposite sense, as shown in FIG. 11c. The barbs on the projections now face proximally. It should be noted that because of the geometry of the bobbin body in relation to the washer, considerably more force would be required on either the inner or outer barbs to “flip” the washer than is required from the projections 119.

The sequence of operation shown in FIGS. 1d and 1e is similar to that of previous embodiments: one full withdrawal of the plunger followed by one full depression of the plunger is permitted, with a degree of free movement provided by the plunger sleeve 117 to allow aspiration of blood or agitation of the contents of a vial.

1. A syringe comprising:
(a) a barrel having a cylindrical interior surface substantially free of discontinuities over its usable extent;
(b) a plunger including a plunger head and a shaft;
(c) a restrictor bobbin movable with respect both to the barrel and the shaft to permit the drawing up and delivery of a fluid over the usable extent of the barrel, whilst limiting further use of the syringe;
(d) the restrictor bobbin and/or shaft together comprising one or more members which are freely movable with respect to each other over a limited distance which is smaller that the usable extent of the barrel so as to permit repeated distal and proximal cycles of movement of the plunger head over the said limited distance.

2. A syringe as claimed in claim 1 wherein the said members which are freely relatively movable to permit repeated movement of the plunger head comprise the complete restrictor bobbin, the complete plunger shaft, or a part or parts of either or any combination thereof.

3. A syringe as claimed in claim 1 wherein the said movable member(s) comprise or may be provided by deformable portions of the bobbin or shaft, which are preferably resiliently deformable.

4. A syringe as claimed in claim 1 wherein the said repeated movement of the plunger head is permitted at least when the plunger head is at or adjacent a proximal end of the said usable extent of the barrel.

5. A syringe as claimed in claim 1, wherein the said repeated movement of the plunger head is permitted at substantially every relative position of the plunger and barrel over the said usable extent of the barrel.

6. A syringe as claimed in claim 1 wherein the said free movement of the bobbin and shaft with respect to each other is permitted in a first region of the shaft and resisted in a second region of the shaft.

7. A syringe as claimed in claim 1, wherein the distance over which the said repeated movement of the plunger head is possible is between 0.1 and 50% of the said barrel usable extent, preferably between 1 and 20%.

8. A syringe as claimed in claim 1, wherein the said distance over which repeated movement of the plunger head is possible corresponds to a swept volume which is between 10 and 2,000 microlitres, preferably between 50 and 1,000 microlitres, more preferably between 100 and 500 microlitres.

9. A syringe as claimed in claim 1, wherein the said distance over which repeated movement of the plunger head is possible is 0.5 mm to 20 mm, preferably 1 mm to 15 mm, more preferably 1.5 mm to 10 mm, still more preferably 2 mm to 7 mm or about 3 mm or 4 mm.

10. A syringe as claimed in claim 1 wherein the restrictor bobbin incorporates a projection, tine, tang, barb, serration or other like formation or member in engagement with the barrel interior wall to resist motion in one direction and permit motion in the opposite direction.

11. A syringe as claimed in claim 1 wherein the plunger is provided with formations for restricting the movement of the restrictor bobbin with respect to the plunger shaft.

12. A syringe as claimed in claim 1 wherein the plunger shaft is formed with a first region over which unrestricted movement of the shaft with respect to the restrictor bobbin is possible and a second region over which movement of the shaft with respect to the restrictor bobbin is substantially restricted to unidirectional movement.

13. A syringe as claimed in claim 1 wherein the restrictor bobbin comprises two or more relatively slidable or relatively deformable parts.

14. A syringe as claimed in claim 1 contained in a sterile pack with its plunger substantially fully depressed.

15. A syringe as claimed in claim 1 contained in a sterile pack with its plunger refracted, preferably to substantially its full extent.

16. A syringe as claimed in claim 15 wherein the syringe contains diluent liquid.

17. A syringe as claimed in claim 14 wherein the bobbin incorporates a restrictor member for restricting movement of the plunger with respect to the bobbin or the bobbin with respect to the barrel, and wherein the direction in which such movement is restricted is reversible.
